(19) United States
(12) Patent Application Publication
(10) Pub. No.: US 2026/0337222 A1
(43) Pub. Date: Oct. 29, 2026

(54) DETERGENT AND METHOD FOR PURIFYING A BIOTHERAPEUTIC

(71) Applicant: MEDIMMUNE, LLC, Gaithersburg, MD (US)

(72) Inventors: Alan HUNTER, Gaithersburg, MD (US); Matthew ASPELUND, Gaithersburg, MD (US); Dhanesh GADRE, Gaithersburg, MD (US); Thomas LINKE, Gaithersburg, MD (US); Kamiyar REZVANI, Gaithersburg, MD (US); Guoling XI, Gaithersburg, MD (US)

(21) Appl. No.: 19/721,555

(22) Filed: Jun. 26, 2026

Related U.S. Application Data
(63) Continuation of application No. 18/001,637, filed on Jun. 29, 2021.
(60) Provisional application No. 63/046,989, filed on Jul. 1, 2020.

(51) Int. Cl. C07K 1/36

(52) U.S. Cl. CPC C07K 1/36

(57) ABSTRACT

Provided herein is an environmentally compatible detergent for use in purification of a recombinantly produced biotherapeutic. The environmentally compatible detergent includes Laureth-9 and can be used for viral clearance, cell lysis, and removal of impurities such as host cell proteins an endotoxins. Advantageously, Laureth-9 does not adversely impact product quality.

### FIELD OF THE INVENTION

Provided herein is an environmentally compatible detergent for use in purification of a biologic therapeutic. The environmentally compatible detergent is suitable for viral clearance, cell lysis, and removal of impurities such as host cell proteins an endotoxins and does not adversely impact product quality.

### BACKGROUND

Biotherapeutics, which include macromolecule therapeutics, such as polypeptides, proteins, antibodies, polynucleotides and viral vectors are a rapidly growing portion the therapeutics available to medical practitioners for treatment of a wide range of diseases and disorders. One reason for the success of biotherapeutics is the high specificity towards targets and superior safety profile as compared to many small molecule therapeutics.

Biotherapeutics are typically produced using biological materials such as cell lines, cell culture fluids, and tissue or body fluids and have a risk of virus contamination, for example, due to a contaminated cell bank (endogenous contamination), or by viral introduction during production (adventitious contamination).

Viruses contain DNA or RNA, single stranded or double stranded, enveloped or non-enveloped. The infectious unit of a non-enveloped virus, the viron, includes a capsid protein and nucleic acid (DNA or RNA). Enveloped viruses include a lipid bilayer that encases the capsid, which includes virus-encoded membrane-associated proteins.

Biotherapeutic manufacturing processes typically include steps to remove viruses from the product stream as well as other impurities such as host cell proteins an endotoxins. Viral clearance processes can include physical removal and/or virus inactivation steps. Physical removal is generally accomplished by virus filtration or column chromatography. Viral inactivation refers to processes that cause an irreversible disruption or denaturation of the viral structure and can be achieved by incubating the product stream with a solvent or detergent, by heating the product stream (pasteurization), or by exposure to low (acidic) pH. Often purification processes include both physical removal and chemical inactivation processes to increase viral clearance.

Viruses encapsulated by an outer membrane (enveloped viruses) are generally susceptible to solvents/detergents and exposure to low pH, while non-enveloped viruses with an outer protein capsid are more physiochemically resistant and generally require physical removal. Conley et al. (2017) “Evaluation of Eco-Friendly Zwitterionic Detergents for Enveloped Virus Inactivation,” Biotech. Bioeng. 114(4):813-820.

Triton X-100 is a nonionic detergent that has been commonly used by manufacturers in the production of commercial biotherapeutics for over 30 years because it is effective at viral inactivation, typically achieving greater than 4 logs of enveloped virus inactivation under a diverse set of experimental conditions. Conley et al. (2017) “Evaluation of Eco-Friendly 10 Zwitterionic Detergents for Enveloped Virus Inactivation,” Biotech. Bioeng. 114(4):813-820. However, recent ecological studies have suggested that Triton X-100 and octlyphenol, a Triton X-100 degradation product, can behave as endocrine disrupters in aquatic organisms, raising concerns from an environmental impact perspective. Conley et al. (2017) “Evaluation of Eco-Friendly Zwitterionic Detergents for Enveloped Virus Inactivation,” Biotech. Bioeng. 114(4):813-820. In 2012, the European Chemicals Agency (ECHA) included Triton X-100 in the Candidate List of substances of very high concern (SVHC) of the Registration, Evaluation, Authorization and Restriction of Chemicals (REACH) Regulation. In 2019, the ECHA included Triton X-100 in the Authorization List (Annex XIV) and mandated that Triton X-100 be replaced in manufacturing processes by a “sunset date” of Jan. 4, 2021.

U.S. Pat. No. 10,611,795 (METHODS FOR VIRAL INACTIVATION USING ECO-FRIENDLY DETERGENTS) describes the use of an environmentally compatible detergent, Triton CG-110, as alternative to Triton X-100 for viral inactivation.

International Patent Publication No. WO 2019/055463 (METHODS OF INACTIVATION OF VIRUSES USING N-METHYLGLUCAMIDE AND ITS DERIVATIVES) describes the use of N-methylglucamides as an alternative to Triton X-100 for viral inactivation.

Lauryldimethylamine N-oxide (LDAO) is an eco-friendly zwitterionic detergent alternative to Triton X-100 that has also been evaluated for enveloped virus inactivation. See, Conley et al. (2017) “Evaluation of eco-friendly zwitterionic Detergents for Enveloped Virus Inactivation” Biotech. and Bioeng. 114(4):813-820.

However, there remains a need for alternative detergents that are environmentally friendly and effective for viral inactivation during manufacturing of commercial biotherapeutics.

### SUMMARY

Provided herein is an environmentally compatible detergent for use in manufacturing a biotherapeutic of interest. In particular, an environmentally compatible detergent is provided for use in purification of a recombinantly produced biotherapeutic. In one aspect, the environmentally compatible detergent is used for viral inactivation, cell lysis, removal of impurities such as host cell proteins and endotoxins, or a combination thereof. In one aspect, the environmentally compatible detergent does not adversely impact product quality. In one aspect, the environmentally compatible detergent includes Laureth-9.

In one aspect, a method for inactivating enveloped virus is provided. In one aspect, a method is provided for inactivating enveloped virus in a mixture that includes a recombinant biotherapeutic of interest. In one aspect, the method includes adding a solution that contains a virus-inactivating amount of Laureth-9 to the mixture and incubating the mixture to inactivate enveloped virus. In one aspect, the solution containing Laureth-9 provides a log reduction value (LRV) greater than about 1 log₁₀, 2 log₁₀, 3 log₁₀ or 4 log₁₀ as compared to a control that does not include a surfactant. In one aspect, the solution containing Laureth-9 provides a log reduction value (LRV) greater than about 1 log₁₀, 2 log₁₀, 3 log₁₀ or 4 log₁₀ as compared to a control that does not include Triton X-100. In one aspect, the solution containing Laureth-9 provides a log reduction value (LRV) greater than about 1 log₁₀, 2 log₁₀, 3 log₁₀ or 4 log₁₀ as compared to a control that does not include Laureth-9. In one aspect, LRV is determined by quantitative PCR or an infectivity assay. In one aspect, LRV is determined by a plaque assay. In one aspect, the solution includes from about 0.1% to about 2% (w/v) Laureth-9. In one aspect, the mixture is incubated with Laureth-9 for at least about 1 minute. In one aspect, the mixture is incubated with Laureth-9 for at least about 5 minutes. In one aspect, the mixture is incubated with Laureth-9 for about 1 minute to about 120 minutes. In one aspect, the mixture is incubated with Laureth-9 at room temperature.

In one aspect, the enveloped virus is a DNA or RNA virus. In one aspect, the enveloped virus is a single stranded or double stranded virus. In one aspect, the enveloped virus includes Herpesviridae, Hepadnaviridae, Togaviridae, Arenaviridae, Flaviviridae, Orthomyxoviridae, Paramyxoviridae, Bunyaviridae, Filoviridae, Coronaviridae, Astroviridae, Bornaviridae, Arteriviridae, or a combination thereof.

In one aspect, a method for lysing cells is provided. In one aspect, a method for lysing cells that express a recombinant biotherapeutic of interest is provided. In one aspect, the method includes contacting a mixture containing the cells with a solution that includes a cell disrupting amount of Laureth-9; and incubating the mixture to lyse the cells. In one aspect, at least about 75%, 80%, 85%, 90%, or 95% of the cells in the mixture are lysed after the mixture is incubated with the Laureth-9 solution. In one aspect, the solution includes from about 0.1% to about 2% (w/v) Laureth-9. In one aspect, the mixture is incubated with Laureth-9 for at least about 1 minute. In one aspect, the mixture is incubated with Laureth-9 for at least about 5 minutes. In one aspect, the mixture is incubated with Laureth-9 for about 1 minute to about 120 minutes. In one aspect, the mixture is incubated with Laureth-9 at room temperature.

In one aspect, a method for removing endotoxin from a mixture that includes a biotherapeutic of interest is provided. In one aspect, the method includes contacting a mixture containing the biotherapeutic of interest with a solution that includes an endotoxin removing amount of Laureth-9; and incubating the mixture. In one aspect, the endotoxin concentration is reduced to less than about 1 EU/mg. In one aspect, the solution includes from about 0.1% to about 2% (w/v) Laureth-9.

In one aspect, the method includes a step of filtering the mixture that includes the recombinant biotherapeutic of interest. In one aspect, filtering is performed before incubating the enveloped virus with the solution that includes Laureth-9. In one aspect, filtering is performed after incubating the enveloped virus with the solution that includes Laureth-9. In one aspect, filtering includes ultrafiltration or depth filtration.

In one aspect, the method includes one or more chromatography steps. In one aspect, one or more chromatography steps include loading a mixture that includes the biotherapeutic of interest onto a chromatography support and washing the chromatography support with a wash solution that includes Laureth-9. In one aspect, the wash solution includes from about 0.1% to about 2% (w/v) Laureth-9.

In one aspect, a method for purifying a biotherapeutic of interest is provided. In one aspect, the method includes loading a mixture that includes the biotherapeutic of interest and one or more impurities onto a chromatography support; washing the chromatography support with a wash solution that includes Laureth-9; and eluting the biotherapeutic of interest from the chromatography support to obtain a purified eluate containing the biotherapeutic of interest.

In one aspect, the wash solution includes a host cell protein-clearing amount of Laureth-9. In one aspect, the purified eluate has a host cell protein content that is reduced relative to a host cell protein content in an eluate from a chromatography support that was not washed with a wash solution that includes Laureth-9. In one aspect, the host cell protein content in the purified eluate from the chromatography support that was washed with a wash solution that includes Laureth-9 is reduced by at least about 50%, 45%, 40%, 35%, 30% or 25% as compared to the host cell content of the mixture applied to the column. In one aspect, the host cell protein content in the purified eluate from the chromatography support that was washed with a wash solution that includes Laureth-9 is less than about 2000 ng HCP/mg protein, 1700 ng/mg, 1500 ng/mg, 1200 ng/mg, 1000 ng/mg, 500 ng/mg, or 100 ng/mg.

In one aspect, the wash solution includes an endotoxin removing amount of Laureth-9. In one aspect, the purified eluate includes an endotoxin concentration below about 1 EU/mg.

In one aspect, the wash solution includes from about 0.1% to about 2% (w/v) Laureth-9.

In one aspect, the recombinant biotherapeutic of interest is a therapeutic macromolecule. In one aspect, the therapeutic macromolecule is a therapeutic polypeptide or a therapeutic polynucleotide. In one aspect, the recombinant biotherapeutic of interest is an enzyme, a soluble receptor, a growth factor, a hormone, a cytokine, an antibody, an antigen-binding antibody fragment, an antibody-drug conjugate, a fusion polypeptide, or a non-enveloped virus. In one aspect, the recombinant biotherapeutic of interest is an antibody or antigen-binding antibody fragment. In one aspect, the antibody is a monoclonal antibody, polyclonal antibody, multivalent antibody, multispecific antibody, chimeric antibody, humanized antibody or human antibody. In one aspect, the non-enveloped virus is an adenovirus or adeno-associated virus (AAV).

In one aspect, the recombinant biotherapeutic of interest is produced by a cell. In one aspect, the recombinant biotherapeutic of interest is produced by a eukaryotic cell. In one aspect, the recombinant biotherapeutic of interest is produced by a prokaryotic cell. In one aspect, the recombinant biotherapeutic of interest is produced by a mammalian cell. In one aspect, the mammalian cell is a Chinese Hamster Ovary (CHO), baby hamster kidney (BHK), murine hybridoma, or murine myeloma cell.

In one aspect, chromatography is selected from one or more of affinity chromatography, ion exchange chromatography hydrophobic interaction chromatography, hydroxyapatite chromatography, and mixed mode chromatography. In one aspect, chromatography includes affinity chromatography. In one aspect, affinity chromatography is selected from protein A chromatography, protein G chromatography, protein L chromatography, or a camelid antibody ligand (V_{H}H) affinity chromatography step. In one aspect, ion exchange chromatography is selected from anion exchange chromatography and cation exchange chromatography.

In one aspect, the method includes a step of filtering a product stream. In one aspect, filtering includes ultrafiltration or depth filtration.

In one aspect, the recombinant biotherapeutic of interest has a bioactivity within about 25%, 15%, 10%, 5%, 4%, 3%, 2%, or 1% of a control recombinant biotherapeutic. In one aspect, the control recombinant biotherapeutic has not been contacted with a Laureth-9.

In one aspect, a method of producing recombinant non-enveloped virus is provided. In one aspect, the method includes culturing a host cell that includes a genome of a non-enveloped virus; and contacting the host cell with a solution that includes Laureth-9. In one aspect, the host cell is contacted with a solution that includes from about 0.1% to about 2% Laureth-9. In one aspect, the host cell is contacted with the solution that includes Laureth-9 for about 1 minute to about 120 minutes. In one aspect, the method includes preparing a cell paste of the host cells expressing the non-enveloped virus and contacting the cell paste with a solution that includes a cell-lysing amount of Laureth-9. In one aspect, the non-enveloped virus is an adenovirus or an adeno-associated virus (AAV). In one aspect, the purified non-enveloped virus has a physical titer greater than about 10 log₁₀ vg/ml. In one aspect, the purified non-enveloped virus has an infectious titer greater than about 8 log₁₀ TCID50/mL. In one aspect, viral infectivity is not significantly reduced compared to a control that is not contacted with a solution that includes Laureth-9.

In one aspect, the method of producing a recombinant non-enveloped virus is provided. In one aspect, the method includes a step of loading a mixture that includes the non-enveloped virus onto a chromatography support; washing the chromatography support with a wash solution that includes Laureth-9; and eluting the non-enveloped virus from the chromatography support to obtain a purified eluate that includes the non-enveloped virus. In one aspect, the wash solution includes from about 0.1% to about 2% (w/v) Laureth-9.

In one aspect, a method of purifying a non-enveloped virus is provided. In one aspect, the method includes contacting a mixture comprising the non-enveloped virus with a solution comprising Laureth-9 and filtering the mixture. In one aspect, filtering is performed before contacting the mixture with the solution that includes Laureth-9. In one aspect, filtering is performed after contacting the mixture with the solution that includes Laureth-9. In one aspect, filtering includes ultrafiltration or depth filtration. In one aspect, the solution includes from about 0.1% to about 2% (w/v) Laureth-9.

### BRIEF DESCRIPTION OF THE DRAWINGS

FIG. 1 shows the Log₁₀ virus reduction obtained with a 1% surfactant solution of Brij L9 (Laureth-9, sourced from Croda International Plc., East Yorkshire, United Kingdom), Polidocanol 600 (Laureth-9, sourced from Scharer & Schlapfer A G, Rothrist, Switzerland), Myrj S25 or Brij S20.

FIG. 2 shows the Log₁₀ virus reduction obtained for two antibody samples (BisAb and BisFusion) using different amounts of Laureth-9 from different sources. BisAb was treated with with a 1% solution of Brij L9 (Laureth-9, sourced from Croda International Plc., East Yorkshire, United Kingdom) or a 1% solution of Polidocanol 600 (Laureth-9, sourced from Scharer & Schlapfer AG, Rothrist, Switzerland). BisFusion was treated with a 1% or 0.1% solution of Brij L9 (Laureth-9 sourced from Croda International Plc). A greater than 4 Log₁₀ inactivation was observed in all samples in 1 minute.

### DETAILED DESCRIPTION

Provided herein are methods for purifying a biotherapeutic using an environmentally compatible detergent.

### DEFINITIONS

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, “a” or “an” may mean one or more. As used herein in the specification and claims, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the present disclosure. Furthermore, compositions, systems, host cells, and/or vectors of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

The term “detergent” refers to an amphiphilic molecule that that possesses a hydrophilic (polar) head group and a hydrophobic (non-polar) tail group. The amphiphilic structure of detergents allow them to interact with other molecules, such as proteins or enveloped viruses, in an aqueous solution. Detergents can be included in biotherapeutic manufacturing processes for a variety of purposes including, for example, as a solubilizing agent; a stabilizing agent, for example, to prevent biomolecule aggregation; for viral inactivation; for removal of impurities, for example, host cell proteins or endotoxins; or a combination thereof. Detergents can be classified into three groups, depending on electrical charge. Anionic detergents include a negatively charged, hydrophilic group. Cationic detergents include a positively charged, hydrophilic group. Non-ionic detergents have no positive or negative charge. Advantageously, non-ionic detergents help retain biomolecule structure and activity. Zwitterionic surfactants, also called amphoteric surfactants, carry both a positive and a negative charge, which can be permanent or dependent on pH.

The critical micelle concentration (CMC) refers to the concentration at and above which the detergent forms micelles. Below the CMC, the surface tension decreases with increasing surfactant concentration. Above the CMC, additional detergent added to the system forms micelles.

The term “surfactant” or “surface active agent” refers to a compound that contains both hydrophobic and hydrophilic groups and is semi-soluble in both organic and aqueous solvents. Surfactants can be non-ionic, cationic, anionic or zwitterionic.

As used herein, “environmentally compatible” refers to a substance which has a low burden or impact on the natural environment. In one aspect, “environmentally compatible” refers to a substance that is in conformance with regulatory guidelines or standards relating to environmental conservation. In one aspect, an “environmentally compatible” substance does not behave as an endocrine disrupter.

The term “bioactive agent” can refer to any substance that is suitable for therapeutic, prophylactic, or diagnostic use and can be used interchangeably with the term “therapeutic agent.” A bioactive agent can include “biotherapeutic” products in which the active substance is obtained from a biological source. Examples of biotherapeutics include macromolecules, for example, therapeutic polynucleotides or polypeptides. Examples of biotherapeutics include, but are not limited to proteins and hormones, monoclonal antibodies (mAbs), cytokines, growth factors, gene therapy products, viral vectors, vaccines, and gene-silencing/editing therapies. Bioactive agents include, but are not limited to, naturally occurring or recombinantly produced macromolecules. In one aspect, the bioactive agent is of therapeutic, scientific or commercial interest.

The term “cell culture” refers to the growth and propagation of host cells outside of a multicellular organism or tissue and includes culturing host cells in suspension and/or attached to a solid substrate. Host cells can be cultured in small scale cultures, for example, in a laboratory setting or in large scale cultures, for example, in a commercial scale bioreactor. Cell culture can include continuous, batch and fed-batch culture systems. Host cells may be cultured, for example, in fluidized bed bioreactors, hollow fiber bioreactors, roller bottles, shake flasks, or stirred tank bioreactors, with or without microcarriers, and operated in a batch, fed batch, continuous, semi-continuous, or perfusion mode.

The terms “polypeptide” and “protein” are used interchangeably herein to refer to polymers of amino acids of any length. The polymer may be linear or branched, and can include modified amino acids, non-natural amino acids or be interrupted by non-amino acids. In one aspect, the terms “polypeptide” and “protein” refer to antibodies or antigen-binding antibody fragments.

The term “polynucleotide” refers to a polymer of ribonucleic acid (RNA), deoxyribonucleic acid (DNA), or a DNA/RNA hybrid polymer. The polynucleotide can be single- or double-stranded and can include sense or antisense polynucleotide sequences of DNA or RNA. The DNA or RNA molecules can include complementary DNA (cDNA), genomic DNA, synthesized DNA, or a hybrid thereof, or an RNA molecule such as mRNA, including untranslated and translated regions. In one aspect, the polynucleotide includes non-canonical nucleotides such as inosine, thiouridine, or pseudouridine or chemically modified nucleotides.

An “isolated” polypeptide, protein, peptide, or polynucleotide is a molecule that has been removed from its natural environment. In one aspect, the “isolated polypeptide” has been recovered from a cell or cell culture from which it was expressed. It is to be understood that “isolated” polypeptides, proteins, peptides, or polynucleotides may be formulated with excipients such as diluents or adjuvants and still be considered isolated.

A “purified” polypeptide, protein, peptide or polynucleotide is a molecule that has been increased in purity, such that it is more pure than in its natural environment and/or when initially produced, synthesized or amplified. Purity is a relative term and does not necessarily require absolute purity.

A “product stream” refers to a material or solution that contains a biotherapeutic molecule of interest during a manufacturing or purification process. Examples of a product stream include, but are not limited to, cell culture media, harvested cell culture fluid (HCCF), or a collected pool containing a biotherapeutic after one or more purification process steps.

In one aspect, the product stream contains one or more impurities. “Impurities” refers to materials present in the product stream that are not the desired biotherapeutic. In one aspect, the product stream contains one or more process-related impurities, including, but not limited to, host cell protein (HCP), host cell DNA, viral contaminants, endotoxins, extractables from resins and filters, and leached Protein A. Viral contaminants can include virus fragments, viral nucleic acids and adventitious or endogenous viruses. In one aspect, product-related impurities include, but are not limited to size variants such as aggregates, or fragments; charge variants; glycosylation variants; or variants due to oxidation, deamidation or denaturation

The term “recombinant” when used in reference to a polynucleotide, peptide, polypeptide, or protein means of, or resulting from, a combination of material that is not known to exist in nature. A recombinant molecule can be produced by any of the well-known techniques available in the field of recombinant technology, including, but not limited to, polymerase chain reaction (PCR), gene splicing (e.g., using restriction endonucleases), and solid-phase synthesis of nucleic acid molecules, peptides, or proteins. The term “recombinantly-expressed” refers to a protein of interest is expressed in a “recombinant host cell” that has been genetically altered, or is capable of being genetically altered, by introduction of an exogenous polynucleotide, such as a recombinant plasmid or vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell” as used herein.

“Viral clearance” refers to removal or inactivation of virus from a mixture, for example, from a product stream containing a biotherapeutic of interest. Virus removal refers to a decrease in the number of viral particles in a sample and can be accomplished by methods such as affinity chromatography or filtration. “Viral inactivation” refers a process in which a virus contained in a composition is rendered nonfunctional. Methods of virus inactivation are known and include, for example, heat activation, pH inactivation, and chemical inactivation, for example, using a surfactant.

“Log₁₀ reduction value” or “LRV” refers to a calculated ratio between viral titer in the starting material and in the relevant product fraction. LRV can be used to describe the capacity of a process step to remove or inactivate virus. LRV can be determined using a known model virus such as murine leukemia virus (MuLV) or minute virus of mice (MVM) and calculated by quantitative PCR (qPCR) or using an infectivity assay.

The term “biological activity” refers to a function of a molecule and can encompass, biological function, biochemical function, physical function, and chemical function. Examples of biological activity include, but are not limited to, enzymatic activity; ability to interact with or bind to another molecule; ability to activate, promote, stabilize, inhibit, suppress, or destabilize a function of another molecule; and ability to localize to a specific position in a cell. As used herein, “biological function,” with regard to a polynucleotide, for example, a gene or polypeptide related thereto, can refer to a specific function that the polynucleotide, gene, or polypeptide can have in a living body. Examples include, but are not limited to, production of a specific protein, enzymatic activity, impartation of resistance, and the like. Biological activity can be measured using technique known in the art. Biological activity can be evaluated using in vitro, in vivo and/or in situ assays indicative of activity for a particular biotherapeutic.

Filtration refers to a process by which particles in a liquid suspension are separated from the liquid by passing the liquid through the pores of a filter. The liquid which passes through the filter is referred to as the filtrate. The retentate is the portion that is retained by the filter.

### Overview

Provided herein is an environmentally compatible detergent for use in the manufacture of a biotherapeutic. In one aspect, the biotherapeutic is recombinantly produced. The environmentally compatible detergent can be used in any step during the manufacturing process. In one aspect, the environmentally compatible detergent is used during a downstream purification process. In one aspect, the environmentally compatible detergent is used during a purification process for a biotherapeutic of interest. In one aspect, an environmentally compatible detergent is provided for viral clearance, cell lysis, and removal of impurities such as host cell proteins and endotoxins. In one aspect, the environmentally compatible detergent does not adversely impact product quality.

Advantageously, the product quality of the biotherapeutic is maintained after contacting the environmentally compatible detergent. In one aspect, the bioactivity of the biotherapeutic is within about 25%, 15%, 10%, 5%, 4%, 3%, 2%, or 1% of the activity a control biotherapeutic, for example, a control biotherapeutic that has not been contacted with a surfactant In one aspect, the bioactivity of the biotherapeutic is within about 25%, 15%, 10%, 5%, 4%, 3%, 2%, or 1% of the activity a control biotherapeutic, for example, a control biotherapeutic that has not been contacted with a Triton X-100. In one aspect, the bioactivity of the biotherapeutic is within about 25%, 15%, 10%, 5%, 4%, 3%, 2%, or 1% of the activity a control biotherapeutic, for example, a control biotherapeutic that has not been contacted with a Laureth-9. Methods for determining biological activity of a biotherapeutic are known and include, for example, in vitro, in vivo and/or in situ assays indicative of activity for a particular biotherapeutic.

Non-ionic detergent alternatives to Triton X-100 were evaluated based on hydrophilic-lipophilic balance (HLB), water solubility, viral inactivation kinetics, availability, safety and environmental compatibility. Surprisingly, Laureth-9, an environmentally compatible non-ionic surfactant, demonstrated process performance and product quality equivalent to Triton X-100 for viral inactivation, cell lysis, host cell protein (HCP) clearance, and endotoxin clearance, in contrast to the findings of Conley et al. (2017) “Evaluation of eco-friendly zwitterionic Detergents for Enveloped Virus Inactivation” Biotech. and Bioeng. 114(4):813-820, who concluded that Brij 35 (polyoxyethylene (23) lauryl ether), a surfactant that is structurally related to Laureth-9 but having a longer polyoxyethylene chain, was not effective for viral inactivation.

### Laureth-9

In one aspect, an environmentally compatible detergent is provided for use in purifying a biotherapeutic. In one aspect, the biotherapeutic is recombinantly produced. In one aspect, the environmentally compatible detergent includes Laureth-9.

Laureth-9 (CAS Numbers: 3055-99-0; 9002-92-0; 68439-50-9) is a non-ionic detergent (HLB-value of approximately 13.3) with a chemical formula of C₃₀H₆₂O₁₀ (due to its polymeric properties the average empirical formula is shown); an average molecular weight of 580 g/mol; and has the IUPAC chemical name 3,6,9,12,15,18,21,24,27-nonaoxanonatriacontan-1-ol. Formula 1, below, provides a typical structure for Laureth-9.

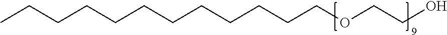

Laureth-9 can be manufactured by the reaction of an alcohol and ethylene oxide, which typically produces a mixture of ethoxylates with different numbers of ethylene oxide units. Laureth-9 has an average alkyl chain of 12 to 14 carbon atoms (C₁₂₋₁₄) and an average ethylene oxide chain of 9 ethylene oxide units (EO₉).

Laureth-9 is also referred to as Brij L9; Polidocanol 600; Polydocanol; Polyoxyethylene (9) lauryl ether; Polyethylene glycol 450 lauryl ether; PEG-9 lauryl ether; Nonaoxyethylene monododecyl ether; Nonaethylene glycol monododecyl ether; Nonaethylene glycol monolauryl ether; Lauromacrogol 400; Macrogol 9 lauryl ether; Polyoxyl 9 lauryl ether; Aethoxy-sklerol; Polyoxyl 9 lauryl ether; and Macrogol Lauryl Ether 9.

In one aspect, a solution is provided that includes Laureth-9. In one aspect, the solution includes Laureth-9, a solvent, and one or more other agents such as chelating agents or preservatives. In one aspect, a solution is provided that includes Laureth-9 and one or more additional detergents. In one aspect, the solution does not include Triton X-100. In one aspect, a solution is provided that includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9.

### Viral Inactivation

Viral clearance is one goal of downstream purification processes. Methods for viral clearance include physical removal of viral particles and viral inactivation. Methods for removal of viral particles include filtration, which can remove both enveloped and non-enveloped viruses. Methods for viral inactivation include heat (pasteurization), low pH inactivation and/or detergent treatment. In one aspect, one or more viral inactivation methods are used to reduce levels of enveloped viruses in a product stream containing a biotherapeutic of interest.

In one aspect, a method is provided for virus inactivation. In one aspect, a method for inactivating an enveloped virus is provided. In one aspect, viral inactivation is performed in a mixture that includes a biotherapeutic of interest. In one aspect, a method for inactivating an enveloped virus during manufacture of a biotherapeutic of interest is provided. In one aspect, the mixture includes a product stream from a purification process for a biotherapeutic of interest. In one aspect, the product stream includes, but is not limited to, cell culture media, harvested cell culture fluid (HCCF), a capture pool, a flow through pool, or a filtrate.

An enveloped virus is a virus that includes an outer envelope that is typically derived from portions of a host cell membrane, which also but includes some viral glycoproteins. Glycoproteins on the surface of the envelope help the virus enter a host cells by binding to receptor sites on the host cell membrane. The virus infects a host cell by fusion of the viral envelope with the host cell membrane. The lipid bilayer envelope of viruses is relatively sensitive to detergents. In one aspect, an environmentally compatible detergent is provided that inactivates enveloped viruses by damaging, for example, lysing the viral envelope.

Enveloped viruses include DNA and RNA viruses and can be single-stranded or double-stranded. Examples of enveloped viruses include, but are not limited to, Herpesviridae such as herpes simplex virus, varicella-zoster virus, cytomegalovirus, and Epstein-Barr virus; Hepadnaviridae, for example, Hepatitis B virus; Togaviridae, for example, Rubella virus; Arenaviridae, for example, lymphocytic choriomeningitis virus; Flaviviridae, including, for example, dengue virus, hepatitis C virus, and yellow fever virus; Orthomyxoviridae, for example, influenzavirus A, Influenzavirus B, Influenzavirus C, isavirus, and thogotovirus; Paramyxoviridae, such as measles virus, mumps virus, respiratory syncytial virus, rinderpest virus, and canine distemper virus; Bunyaviridae, such as California encephalitis virus, and Hantavirus; Filoviridae, such as Ebola virus, and Marburg virus; Coronaviridae, such as corona virus; Astroviridae, for example, Astrovirus; Bornaviridae, for example, borna disease virus; and Arteriviridae, including, for example, arterivirus, and equine arteritis virus.

In one aspect, enveloped virus is inactivated by incubating a mixture that includes a biotherapeutic of interest with an environmentally compatible detergent. In one aspect, the environmentally compatible detergent includes Laureth-9. In one aspect, the environmentally compatible detergent includes a virus-inactivating amount of Laureth-9. A “virus-inactivating amount” with reference to an environmentally compatible detergent refers to an amount, such as a concentration, volume or weight ratio, of detergent that is effective to solubilize at least a portion of membrane proteins forming a protein coat around an enveloped virus, resulting in lysis of the virus. In one aspect, a “virus-inactivating amount” refers to an amount of detergent that damages and/or lyses the protein coat of a virus, without adversely affecting the biotherapeutic of interest. In one aspect, a mixture that contains a biotherapeutic of interest is contacted with a solution that includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9. In one aspect, the mixture is incubated with the solution containing Laureth-9 for about 1 minute to about 120 minutes. In one aspect, the mixture is incubated with the solution containing Laureth-9 for at least about 1, 2, 3, 4, 5, 10, 15, 20, 25 or 30 minutes and up to about 60, 90 or 120 minutes. In one aspect, the mixture is incubated with the solution containing Laureth-9 for up to about 1 hour, 2 hour, 3 hours 4 hours, 5 hours, 24 hours or 48 hours. In one aspect, the mixture is incubated with the solution containing Laureth-9 for about 1, 2, 3, 4, 5, 10, 15, 20, 25, 30, 60, 90 or 120 minutes. In one aspect, the mixture is incubated with the solution containing Laureth-9 for about 1 hour, 2 hour, 3 hours 4 hours, 5 hours, 24 hours or 48 hours. In one aspect, the mixture is incubated with Laureth-9 for about 1 minute. In one aspect, the mixture is incubated with Laureth-9 for at least about 5 minutes. In one aspect, the mixture is incubated with Laureth-9 at a temperature from about 4 °C to about 42 °C. In one aspect, the mixture is incubated with Laureth-9 at a temperature from about 4 °C to about 25 °C. In one aspect, the mixture is incubated with Laureth-9 at room temperature, for example, at a temperature of about 20 °C to about 25 °C. In one aspect, the mixture is contacted with a solution that includes Laureth-9 at a pH from about pH 4.5 to about pH 8.5. In one aspect, the mixture is contacted with a solution that includes Laureth-9 at a pH of about 4.5, 5.0, 5.5, 6.0, 6.5, 7.0, 7.5, 8., or 8.5.

In one aspect, viral inactivation is expressed as log reduction value (LRV). In one aspect, the log reduction value can be calculated as LRV=log₁₀×(total virus in product stream following treatment with Laureth-9/total virus in product stream before treatment with Laureth-9). In one aspect, the viral log reduction value (LRV) provided by the detergent solution is greater than about 1 log₁₀. In one aspect, the LRV is greater than about 1 log₁₀, 2 log₁₀, 3 log₁₀ or 4 log₁₀ as compared to a control that is not incubated with a surfactant. In one aspect, the LRV is greater than about 1 log₁₀, 2 log₁₀, 3 log₁₀ or 4 log₁₀ as compared to a control that is not incubated with a surfactant that includes Triton X-100. In one aspect, the LRV is greater than about 1 log₁₀, 2 log₁₀, 3 log₁₀ or 4 log₁₀ as compared to a control that is not incubated with a surfactant that includes Laureth-9. Methods for determining LRV and known and include, for example, quantitative PCR or an infectivity assay. In one aspect, LRV is determined by a plaque assay. In one aspect, viral inactivation can be determined by spiking a sample with a test virus such as X-MuLV, incubating the sample and determination of viral inactivation, for example using a plaque assay.

In one aspect, the method includes a step of filtering the mixture that contains the biotherapeutic of interest. In one aspect, filtering is performed before incubating the enveloped virus with a solution that includes Laureth-9. In one aspect, filtering is performed after incubating the enveloped virus with a solution that includes Laureth-9. In one aspect, filtering includes ultrafiltration. In one aspect, filtering includes depth filtration.

Viral inactivation using a detergent such as Laureth-9 can be performed at any step during the manufacturing process. In one aspect, viral inactivation includes contacting a product stream from a purification process for a biotherapeutic with a solution that includes Laureth-9. In one aspect, cell culture media is contacted with a solution that includes Laureth-9. In one aspect, a harvested cell culture media is contacted with a solution that includes Laureth-9. In one aspect, a capture pool from a chromatography resin that contains biotherapeutic of interest is contacted with a solution that includes Laureth-9. In one aspect, a flow through pool from a chromatography resin that contains a biotherapeutic of interest is contacted with a solution that includes Laureth-9. In one aspect, a filtrate that contains a biotherapeutic of interest is contacted with a solution that includes Laureth-9. In one aspect, a retentate that contains a biotherapeutic of interest is contacted with a solution that includes Laureth-9.

### Cell Culture

In one aspect, the biotherapeutic is produced in a cell culture. In one aspect, the biotherapeutic is expressed from a gene that is endogenous to a host cell. In one aspect, the biotherapeutic is expressed from a gene that is introduced into a host cell, for example, through genetic engineering. In one aspect, the biotherapeutic may be one that occurs in nature. In one aspect, the biotherapeutic may be recombinantly produced or engineered. In one aspect the biotherapeutic is assembled from segments that individually occur in nature. In one aspect, the biotherapeutic includes one or more segments that are not naturally occurring.

In one aspect, the biotherapeutic is produced in an adherent cell culture. In one aspect, the biotherapeutic is produced in a suspension cell culture. In one aspect, the biotherapeutic is produced in a prokaryotic cell. In one aspect, the prokaryotic cell includes Escherichia coli. In one aspect, the biotherapeutic is produced in a eukaryotic cell. In one aspect, the biotherapeutic is produced in an animal cell. In one aspect, the biotherapeutic is produced in a yeast cell. In one aspect, the yeast cell is selected from Saccharomyces cerevisiae, Hansenula polymorpha, Pichia pastoris and Yarrowia lipolytica. In one aspect, the biotherapeutic is produce in an insect cell. In one aspect, the insect cell is a Spodoptera frugiperda (Sf9) insect cell. In one aspect, the biotherapeutic is produced in a mammalian cell. In one aspect, the mammalian cell is selected from human retinoblasts (PER.C6); human embryonic kidney cells (HEK-293); baby hamster kidney cells (BHK); Chinese hamster ovary cells (CHO); mouse sertoli cells (TM4); monkey kidney cells (CV1); African green monkey kidney cells (VERO-76); human cervical carcinoma cells (HeLa); canine kidney cells (MDCK); buffalo rat liver cells (BRL); human lung cells (W138); human liver cells (Hep G2); mouse mammary tumor cells (MMT); TRI cells (Mather et al., Annals N.Y. Acad. Sci., 383:44-68 (1982)); MRC 5 cells; FS4 cells; and a human hepatoma line (Hep G2). In one aspect, the mammalian cell is a Chinese Hamster Ovary (CHO) cell. In one aspect, the cell is a hybridoma cell.

In one aspect, the biotherapeutic is secreted into the cell culture media. In one aspect, the biotherapeutic is expressed in the host cell cytoplasm. In one aspect, the biotherapeutic is expressed in the host cell membrane. In one aspect, the host cell is lysed to release the biotherapeutic. In one aspect, the cell culture media is clarified, for example, by centrifugation, to remove cells and cell debris to form a clarified cell culture media. In one aspect, the cell culture media is purified, for example, using one or more chromatography steps, one or more filtration steps, or a combination thereof.

### Cell Lysis

In one aspect, a method for lysing a host cell is provided. In one aspect, the host cell is in a cell culture. In one aspect, the host cell is in a cell slurry. In one aspect, the host cell is lysed by contacting the cell with a non-ionic surfactant. In one aspect, the host cell is lysed by contacting the cell with Laureth-9. In one aspect, the host cell is lysed by contacting the cell with a solution that includes Laureth-9. In one aspect, the host cell is lysed by contacting a mixture containing the cell with a cell-disrupting amount of Laureth-9. A “cell-disrupting amount” with reference to an environmentally compatible detergent refers to an amount, such as a concentration, volume or weight ratio, of detergent that is effective to rupture a cell membrane and release at least some, or all of the cell contents. In one aspect, a “cell-disrupting amount” refers to an amount of detergent that disrupts a cell membrane and releases all or part of the cell contents, without adversely affecting the biotherapeutic of interest.

In one aspect, the host cell is lysed by contacting a mixture containing the cell with a solution that includes from about 0.1% to about 2% Laureth-9. In one aspect, the host cell is lysed by contacting a mixture containing the cell with a solution that includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the host cell is lysed by contacting a mixture containing the cell with a solution that includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9. In one aspect, the mixture containing the cell is incubated with Laureth-9 for about 1 minute to about 120 minutes. In one aspect, the mixture is incubated with Laureth-9 for at least about 1, 2, 3, 4, 5, 10, 15, 20, 25 or 30 minutes and up to about 60, 90 or 120 minutes. In one aspect, the mixture is incubated with the solution containing Laureth-9 for up to about 1 hour, 2 hour, 3 hours 4 hours, 5 hours, 24 hours or 48 hours. In one aspect, the mixture is incubated with Laureth-9 for about 1, 2, 3, 4, 5, 10, 15, 20, 25, 30, 60, 90 or 120 minutes. In one aspect, the mixture is incubated with the solution containing Laureth-9 for about 1 hour, 2 hour, 3 hours 4 hours, 5 hours, 24 hours or 48 hours. In one aspect, the mixture is incubated with Laureth-9 for about 1 minute. In one aspect, the mixture is incubated with Laureth-9 for at least about 5 minutes. In one aspect, the mixture is incubated with Laureth-9 at a temperature from about 4 °C to about 42 °C. In one aspect, the mixture is incubated with Laureth-9 at a temperature from about 4 °C to about 25 °C. In one aspect, the mixture is incubated with Laureth-9 at room temperature, for example, at a temperature of about 20 °C to about 25 °C. In one aspect, the mixture is contacted with a solution that includes Laureth-9 at a pH from about pH 4.5 to about pH 8.5. In one aspect, the mixture is contacted with a solution that includes Laureth-9 at a pH of about 4.5, 5.0, 5.5, 6.0, 6.5, 7.0, 7.5, 8.0, or 8.5. In one aspect, at least about 75%, 80%, 85%, 90%, or 95% of the cells in the mixture are lysed after the mixture is incubated with the Laureth-9 solution. In one aspect, at least about 80% of the cells in the mixture are lysed after the mixture is incubated with the Laureth-9 solution.

### Endotoxin Removal

In one aspect, a method is provided for removing endotoxin from a mixture that contains a biotherapeutic of interest. In one aspect, the mixture includes a product stream that contains a biotherapeutic of interest. In one aspect, the method includes contacting a mixture with an endotoxin removing amount of an environmentally compatible detergent. In one aspect, the method includes contacting a product stream with an endotoxin removing amount of an environmentally compatible detergent. In one aspect, the method includes contacting a product stream with an endotoxin removing amount of a solution that includes Laureth-9.

Endotoxins are lipopolysaccharides (LPS) derived from the cell membrane of Gram-negative bacteria and are contaminants that can be found in recombinantly produced biotherapeutic preparations. The presence of small amounts of endotoxin in a biotherapeutic preparation can result in a systemic inflammatory reaction resulting in side effects such as endotoxin shock, tissue injury and death. Endotoxin contamination is found in connection with biotherapeutics that are produced using gram negative bacteria such as Escherichia coli and can be introduced during production processes, for example, by non-sterile process conditions. Many purification methods have been developed for endotoxin removal, including, for example, affinity chromatography, size-exclusion chromatography, membrane ultrafiltration, membrane microfiltration, anion-exchange chromatography, cationic-exchange chromatography, hydroxyapatite, hydrophobic interaction, reverse-phase and thiophilic adsorption. Other methods for endotoxin removal include microfiltration and ultrafiltration. Endotoxins exhibit a significant capability to interact with biomolecules, including for example, biotherapeutics such as biotherapeutic proteins.

In one aspect, an “endotoxin-removing amount” with reference to an environmentally compatible detergent refers to an amount, such as a concentration, volume or weight ratio, of detergent that is effective to dissociate endotoxin from a biotherapeutic of interest in a mixture, for example, a product stream from a purification process. In one aspect, an “endotoxin-removing amount” refers to an amount of detergent that is effective to dissociate endotoxin from a biotherapeutic of interest, without adversely affecting the biotherapeutic of interest.

In one aspect, the method includes contacting a mixture, for example, a product stream, with a solution containing an environmentally compatible detergent in an amount effective to dissociate endotoxin from the biotherapeutic of interest. In one aspect, the method includes contacting a mixture containing a biotherapeutic of interest with a solution containing Laureth-9 in an amount effective to dissociate endotoxin from the biotherapeutic of interest. In one aspect, the method includes contacting the product stream with a solution containing Laureth-9 in an amount effective to dissociate endotoxin from the biotherapeutic of interest. In one aspect, the method includes contacting a mixture with a solution that includes from about 0.1% to about 2% (w/v) Laureth-9. In one aspect, the wash solution includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9.

In one aspect, the method includes filtering a mixture, for example, a product stream from a purification process, to remove endotoxin. In one aspect, the method includes filtering a mixture after the product stream is contacted with the solution containing Laureth-9. In one aspect, the mixture is filtered through a molecular weight cut-off filter with a pore size effective to retain the biotherapeutic of interest and allow the dissociated bacterial endotoxin to pass through.

In one aspect, the method includes one or more chromatography steps. In one aspect, a surfactant such as Laureth-9 is included in a chromatography wash solution to increase endotoxin removal during the chromatography process

### Purification

In one aspect, the cell culture media is harvested and clarified. In one aspect, the clarified cell culture media is subjected to one or more purification processes to separate one or more impurities from the biotherapeutic of interest. In one aspect, the biotherapeutic is retained in a product stream. In one aspect, one or more impurities are discarded in a waste stream.

As used herein the term “mixture” can include a “product stream.” The term “product stream” refers to an output obtained from a cell culture which contains the biotherapeutic product of interest, and can include the result of one or more purification process steps, including, for example, the product stream after centrifugation, after chromatography, after filtration or other after other steps in the purification process and can include, for example, cell culture media, harvested cell culture media, clarified cell culture media, a capture pool eluate from a chromatography resin, a flow through pool from a chromatography resin, supernatant, a filtrate, a retentate, and the like.

In one aspect, a biotherapeutic of interest in a product stream is contacted with an environmentally compatible surfactant that includes Laureth-9 during a purification process for the biotherapeutic of interest. Advantageously, the quality of the biotherapeutic product is maintained during the process. In one aspect, treatment of the product stream in a purification process for a biotherapeutic with an environmentally compatible detergent that includes Laureth-9 does not increase the amount of product variants in the product stream, for example, as compared to a purification process without a surfactant or in a purification process in which Triton X-100 is used as a surfactant. In one aspect, the product variants can include size variants, charge variants, oxidation variants, deamidation variants, glycation variants or variants with altered glycan profiles. In one aspect, the product variants include size variants, for example, due to product degradation or aggregation. Methods for detecting the presence of size variants are known, and include, for example, size exclusion chromatography and gel electrophoresis. In one aspect, treatment of the a product stream in a purification process for a biotherapeutic with an environmentally compatible detergent that includes Laureth-9 results in a change of less than about 5% in product variants out of the total amount of product in the product stream, for example, as compared to a purification process without a surfactant or in which Triton X-100 is used as a surfactant. In one aspect, the environmentally compatible detergent results in a less than about 10%, 9%, 8%, 7%, 6%, 5%, 4%, 3%, 2% or 1% increase in product variants out of the total protein in the product stream compared to a manufacturing process without an environmentally compatible detergent that includes Laureth-9, for example, a process without a surfactant or a process in which Triton X-100 is used as a surfactant.

### Chromatography

In one aspect, the product stream is subjected to one or more chromatography steps to separate impurities from the biotherapeutic, for example, based on charge, hydrophobicity, size or affinity. In column chromatography, a liquid mobile phase is passed over a stationary phase (the chromatography resin) and molecules in the mobile phase are separated based on differing interactions with the stationary phase. For example, the interactions with the stationary phase can be based on molecular size (size exclusion chromatography), charge (ion exchange chromatography), hydrophobicity (hydrophobic interaction chromatography), specific binding interactions (affinity chromatography) or a combination thereof (mixed mode chromatography). One or more types of chromatographic columns can be used in a purification process and the number and sequence of chromatographic columns can vary.

Examples of column chromatography resins include, for example, affinity chromatography, ion exchange column chromatography, mixed mode column chromatography, hydroxyapatite chromatography, and hydrophobic interaction column chromatography. Examples of affinity chromatography resins include protein A, protein G and protein L. Other examples of affinity chromatography resins include resins with a ligand that includes a recombinant camelid single domain antibody. In one aspect, affinity chromatography includes LambaFabSelect or KappaSelect chromatography. Ion exchange chromatography includes anion exchange chromatography and cation exchange chromatography. In one aspect, mixed mode chromatography includes CaptoAdhere chromatography.

Chromatography processes include “flow-through” and “bind and elute” processes. In “flow-through” chromatography processes, the desired biotherapeutic flows through the chromatography and can be collected in a “flow through pool,” while impurities are retained on the chromatography resin. In a “bind and elute” chromatography process, the desired biotherapeutic is retained by the chromatography resin and impurities flow through. The desired biotherapeutic is subsequently eluted from the chromatography resin and can be collected in a “capture pool.”

In one aspect, the product stream includes a flow through pool. In one aspect, the product stream includes a capture pool. In one aspect, the flow through pool or capture pool includes an affinity chromatography pool. In one aspect, the flow through pool or capture pool includes a protein A, a protein G or a protein L pool. In one aspect, the flow through pool or capture pool includes a Capto AVB affinity chromatography pool. In one aspect, the flow through pool or capture pool includes an anion exchange chromatography pool. In one aspect, the flow through pool or capture pool includes cation exchange chromatography pool. In one aspect, the flow through pool or capture pool includes a mixed mode chromatography pool. In one aspect, the flow through pool or capture pool includes a CaptoAdhere pool. In one aspect, the flow through pool or capture pool includes a hydroxyapatite pool. In one aspect, the flow through pool or capture pool includes a hydrophobic interaction column pool.

When used in a bind and elute mode, column chromatography typically includes an equilibration step in which an equilibration buffer that is compatible with the biotherapeutic of interest and the chromatographic resin is passed over the column resin. Generally, from about 5 to about 10 column volumes (CVs) of equilibration buffer is used. The sample is then loaded onto the column. Often a loading buffer that is the same as the equilibration buffer is used. Once the biotherapeutic of interest is immobilized onto the stationary phase of the column, impurities that interact only weakly with the column resin can be removed by washing the column with one or more column volumes of a wash solution. In one aspect, the wash solution has the same composition as the equilibration buffer. In another aspect, the wash solution includes components that help disrupt weak interactions between impurities and the column resin. In one aspect, the column is washed until impurities are undetectable in the eluate. After impurities have been washed off of the chromatography resin, molecules such as the biotherapeutic can be eluted with an elution buffer. Generally, the elution buffer has a composition that is different than that of the equilibration buffer and/or wash solution.

In one aspect, a surfactant is included the wash solution. In one aspect, a surfactant that includes Laureth-9 is included in the wash solution. In one aspect, including Laureth-9 in the wash solution enhances viral clearance from the product stream during chromatography purification processes. In one aspect, including Laureth-9 in the wash solution increases removal of host cell proteins (HCP) from the product stream. In one aspect, including Laureth-9 in the wash solution increases endotoxin removal from the product stream. In one aspect, inclusion of an intermediate wash, after the surfactant-containing wash, but prior to elution of the biotherapeutic of interest from the chromatography resin increases the yield of the biotherapeutic in the eluate, in addition to removing residual surfactant from the chromatography support.

When used in flow through mode, chromatography conditions are selected such that the biotherapeutic of interest will not bind to the chromatography resin, but rather will flow through the column, and in which impurities will remain bound to the column resin. Flow through chromatography processes can include a wash step to collect weakly bound biotherapeutic from the column resin.

In one aspect, a method for purifying a biotherapeutic of interest is provided. In one aspect, the method includes loading a mixture that contains the biotherapeutic of interest and one or more impurities onto a chromatography support. In one aspect, the method includes washing the chromatography support with a wash solution that includes an environmentally compatible detergent. In one aspect, the environmentally compatible detergent includes Laureth-9. In one aspect, the method includes eluting the biotherapeutic of interest from the chromatography support to obtain a purified eluate that includes the biotherapeutic of interest.

In one aspect, the wash solution includes from about 0.1% to about 2% (w/v) Laureth-9. In one aspect, the wash solution includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9. In one aspect, a surfactant such as Laureth-9 is included in a chromatography wash solution to increase viral clearance. In one aspect, a surfactant such as Laureth-9 is included in a chromatography wash solution to increase host cell removal. In one aspect, a surfactant such as Laureth-9 is included in a chromatography wash solution to increase endotoxin removal.

In one aspect, a surfactant such as Laureth-9 is included in a chromatography wash buffer to improve viral clearance. In one aspect, the chromatography resin on which a desired biotherapeutic is retained is washed with a wash solution that includes a surfactant such as Laureth-9. In one aspect, the surfactant-containing wash solution is applied to the column prior to elution of the desired biotherapeutic from the chromatography resin. In one aspect, the chromatography resin is an affinity chromatography resin. In one aspect, the wash solution includes a virus-inactivating amount of Laureth-9. In one aspect, the wash solution includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9.

In one aspect, a surfactant such as Laureth-9 is included in a chromatography wash buffer to increase host cell protein removal from the product stream. In one aspect, the chromatography resin on which a desired biotherapeutic is retained is washed with a wash solution that includes a surfactant such as Laureth-9. In one aspect, the surfactant-containing wash solution is applied to the column prior to elution of the desired biotherapeutic from the chromatography resin. In one aspect, the chromatography resin is an affinity chromatography resin. In one aspect, affinity chromatography resin includes a recombinant camelid single domain antibody. In one aspect, affinity chromatography resin includes LambaFabSelect or KappaSelect. In one aspect, the affinity chromatography resin includes Protein A, Protein G or Protein L chromatography resin.

In one aspect, the wash solution includes a host cell protein-clearing amount of Laureth-9. In one aspect, the purified eluate has a host cell protein content that is reduced relative to a host cell protein content in an eluate from a chromatography support that was not washed with a wash solution that includes Laureth-9. In one aspect, the wash solution includes from about 0.1% to about 2% (w/v) Laureth-9. In one aspect, the wash solution includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9. In one aspect, the host cell protein content in the purified eluate from the chromatography support that was washed with a wash solution that includes Laureth-9 is reduced by at least about 50%, 45%, 40%, 35%, 30% or 25% as compared to the host cell content of the mixture applied to the column. In one aspect, the host cell protein content in the purified eluate from the chromatography support that was washed with a wash solution that includes Laureth-9 is reduced by at least about 30% as compared to the host cell content of the mixture applied to the column. In one aspect, the host cell protein content in the purified eluate from the chromatography support that was washed with a wash solution that includes Laureth-9 is less than about 75%, 70%, 65%, 60%, 55%, or 50% of the host cell protein content in the mixture applied to the column. In one aspect, the host cell protein content in the purified eluate from the chromatography support that was washed with a wash solution that includes Laureth-9 is less than about 65% of the host cell protein content in the mixture applied to the column. In one aspect, the host cell protein content in the purified eluate from the chromatography support that was washed with a wash solution that includes Laureth-9 is less than about 2000 ng/mg, 1700 ng/mg, 1500 ng/mg, 1200 ng/mg, 1000 ng/mg, 500 ng/mg, or 100 ng/mg.

In one aspect, the wash solution includes an endotoxin removing amount of Laureth-9. In one aspect, the purified eluate has an endotoxin content that is reduced relative to an endotoxin content in an eluate from a chromatography support that was not washed with a wash solution that includes Laureth-9. In one aspect, the wash solution includes from about 0.1% to about 2% (w/v) Laureth-9. In one aspect, the wash solution includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9. In one aspect, the endotoxin content in the purified eluate from the chromatography support that was washed with a wash solution that includes Laureth-9 is reduced by at least about 99.0%, 99.1%, 99.2%, 99.3%, 99.4%, 99.5%, 99.6%, 99.7%, 99.8%, or 99.9%, as compared to the endotoxin content of the mixture applied to the column. In one aspect, the endotoxin content in the purified eluate from the chromatography support that was washed with a wash solution that includes Laureth-9 is reduced by at least about 99.9%, as compared to the endotoxin content of the mixture applied to the column. In one aspect, the endotoxin content in the purified eluate from the chromatography support that was washed with a wash solution that includes Laureth-9 is less than about 1%, 0.9%, 0.8%, 0.7%, 0.6%, 0.5%, 0.4%, 0.3%, 0.2%. 0.1%, 0.05% or 0.01% of the endotoxin content in the mixture applied to the column. In one aspect, the endotoxin content in the purified eluate from the chromatography support that was washed with a wash solution that includes Laureth-9 is less than about 0.1% of the endotoxin content in the mixture applied to the column. In one aspect, the endotoxin content in the purified eluate from the chromatography support that was washed with a wash solution that includes Laureth-9 is less than about 0.05% or of the endotoxin content in the mixture applied to the column. In one aspect, the endotoxin content in the purified eluate from the chromatography support that was washed with a wash solution that includes Laureth-9 is less than about 0.01% of the endotoxin content in the mixture applied to the column. In one aspect, the purified eluate has an endotoxin concentration below about 1 EU/mg, 0.9 EU/mg, 0.8 EU/mg, 0.7 EU/mg, 0.6 EU/mg, 0.5 EU/mg, 0.4 EU/mg, 0.3 EU/mg, 0.2 EU/mg or 0.1 EU/mg. In one aspect, the purified eluate has an endotoxin concentration below about 1 EU/mg. In one aspect, the purified eluate has an endotoxin concentration below about 0.5 EU/mg. Methods for determining endotoxin concentration are known and include, for example, limulus amebocyte lysate assay (LAL assay). In one aspect, chromatography includes ion exchange chromatography. In one aspect, chromatography includes cation exchange chromatography.

### Filtration

In one aspect, the purification process includes a filtration step. In one aspect, mixture is filtered to remove viral particles. In one aspect, the mixture is a product stream from a purification process. In one aspect, the purification process includes a filtration step to concentrate the biotherapeutic. In one aspect, filtration includes ultrafiltration. In one aspect, filtration includes diafiltration. In one aspect, filtration includes depth filtration. In one aspect, ultrafiltration, depth filtration, or a combination thereof is used to remove one or more impurities, including, for example, viral particles, from a mixture such as a product stream. In one aspect, the product stream includes cell culture media, clarified cell culture media, a capture pool or a flow through pool. In one aspect, the method includes filtering a product stream after the product stream is contacted with a environmentally compatible detergent. In one aspect, the method includes filtering a product stream after the product stream is contacted with Laureth-9. In one aspect, the method includes filtering a product stream after the product stream is contacted with a solution that includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9. In one aspect, the product stream is filtered at least about 15 minutes to at least about 48 hours after addition of Laureth-9. In one aspect, the product stream is filtered at least about 1 hour to at least about 3 hours after addition of Laureth-9.

### Sample

In one aspect, a sample is contacted with a solution that includes Laureth-9. In one aspect, the sample is a product stream from a purification process for a biotherapeutic. In one aspect, the sample is a product stream from a purification process for a recombinantly produced biotherapeutic. In one aspect, the sample is contacted with a solution that includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the sample is contacted with a solution that includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9.

In one aspect, the sample includes harvested cell media. In one aspect, the sample includes clarified cell harvest media. Any conventional means to harvest cells from a cell culture media can be used, including, but not limited to centrifugation or microfiltration. In one aspect, the sample includes whole cells. In one aspect, the sample includes a cell lysate. In one aspect, the sample includes a cell paste slurry formed by concentrating and purifying a biotherapeutic from a cell lysate and cell culture media. In one aspect, the cell paste slurry is formed by pelleting cells from a cell culture and removing the supernatant to form the cell paste. In one aspect, the sample includes a capture pool from a chromatography resin. In one aspect, the sample includes a flow through pool from a chromatography resin. In one aspect, the sample includes a filtrate. In one aspect, the sample includes a retentate.

### Active Agent

The methods described herein can be used in processes for manufacturing a wide variety of compositions, including, for example, compositions having pharmaceutical, diagnostic, agricultural, and/or any of a variety of other properties that are useful in commercial, experimental or other applications. In one aspect, the method is used in a process for manufacturing a therapeutic agent. In one aspect, the method is used in a process for manufacturing a biotherapeutic. In one aspect, the biotherapeutic is naturally occurring. In one aspect, the biotherapeutic is recombinantly produced. In one aspect, the biotherapeutic is homologous to the host cell. In one aspect, the biotherapeutic is heterologous, i.e., exogenous or foreign, to the host cell.

In one aspect, the biotherapeutic includes a therapeutic macromolecule, for example, a therapeutic polynucleotide or polypeptide. In one aspect, the bioactive agent includes a chemically modified macromolecule, for example, a pegylated macromolecule or a macromolecule to which a diagnostic, targeting or therapeutic moiety has been appended. In one aspect, the bioactive agent is of therapeutic, scientific or commercial interest.

In one aspect, the bioactive agent includes a therapeutic protein such as an enzyme or enzymatically active polypeptide, a soluble receptor or receptor ligand, hormone, neurotransmitter, coagulation/clotting factor, growth factor, integrin, cytokine, regulatory factor, interferon, an antigen, a secreted protein, or a fragment thereof. In one aspect, the bioactive agent includes an antibody, antigen-binding antibody fragment, Fc fusion protein, antibody-drug conjugate (ADC), immunoadhesin, or an antigen-binding agent. In one aspect, the biotherapeutic includes gene therapy products; vaccines, including viral-based vaccines; and non-enveloped viral therapeutics. In one aspect, the therapeutic protein includes a fusion protein or a proteolysis targeting chimera (protac). In one aspect, the bioactive agent includes a therapeutic peptide. As used herein “therapeutic peptide” refers to a bioactive polypeptide that includes less than about 500, 250, 100, 50 or 20 amino acids and has a molecular weight below about 100 kDa, or 50 kDa.

In one aspect, the recombinant biotherapeutic of interest is an antibody or antigen-binding antibody fragment. In one aspect, the bioactive agent includes an antibody-like protein. As used herein, the terms “antibody” and “immunoglobulin” can be used interchangeably and refer to a polypeptide or group of polypeptides that include at least one binding domain that is formed by the folding of polypeptide chains having three-dimensional binding spaces with internal surface shapes and charge distributions complementary to the features of an antigenic determinant of an antigen. A naturally occurring antibody or antigen binding fragment thereof typically has a tetrameric form, with two pairs of polypeptide chains, each pair having one “light” and one “heavy” chain. The variable regions of each light/heavy chain pair form an antibody binding site. Each light chain is linked to a heavy chain by one covalent disulfide bond the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains (CH) and each light chain has a variable domain at one end (VL) and a constant domain (CL) at its other end, wherein the constant domain of the light chain is aligned with the first constant domain of the heavy chain and the light chain variable domain is aligned with the variable domain of the heavy chain. Light chains are classified as either lambda chains or kappa chains based on the amino acid sequence of the light chain constant region. Immunoglobulin molecules can be of any isotype (e.g., IgG, IgE, IgM, IgD, IgA and IgY), subisotype (e.g., IgG1, IgG2, IgG3, IgG4, IgAQ1 and IgA2) or allotype (e.g., Gm, e.g., Glm(f, z, a or x), G2m(n), G3m(g, b, or c), Am, Em, and Km(1, 2 or 3)).

The term “antigen-binding fragment” refers to fragments of an antibody that contain at least one antigen-binding site and retain the ability to specifically bind to an antigen. Examples of antigen-binding antibody fragments that can be recombinantly produced include, but are not limited to, fragments that include variable heavy- and light-chain domains, such as single-chain Fvs (scFv), single-chain antibodies, Fab fragments, Fab′ fragments, F(ab′)₂ fragments, dimeric variable region (Diabody) and disulphide-linked variable region (dsFv). Antibody fragments can also include epitope-binding fragments or derivatives of any of the antibodies enumerated above.

The antibody may be oligoclonal, polyclonal, monoclonal, chimeric, camelised, CDR-grafted, multi-specific, bi-specific, catalytic, humanized, fully human, anti-idiotypic, and intrabodies, as well as a recombinantly produced fragment, including a fragment that exhibits a desired biological activity such as an epitope-binding fragment, variants or derivatives thereof, either alone or in combination with other amino acid sequences.

In one aspect, the bioactive agent includes the fragment crystallizable (Fc) region or domain of an IgG, which includes a paired set of antibody heavy chain domains, each of which include a heavy chain constant domain 2 (CH2) and a heavy chain constant domain 3 (CH3), which form a structure of about 50 kDa. The Fc region interacts with cell surface receptors called Fc receptors and some proteins of the complement system, allowing an antibody to activate the immune system.

In one aspect, the biotherapeutic is a polynucleotide. In one aspect, the biotherapeutic is a viral vector. In one aspect, the biotherapeutic includes a non-enveloped virus. In one aspect, the biotherapeutic includes a viral vector such as an adenoviral or adeno-associated viral (AAV) vector.

### Non-Enveloped Virus Biotherapeutic

In one aspect, the biotherapeutic is a non-enveloped virus. In one aspect, the biotherapeutic is a single-stranded non-enveloped virus. In one aspect, the biotherapeutic is an adenovirus. In one aspect, the biotherapeutic is an adeno-associated virus (AAV). In one method a method for purification of a non-enveloped virus is provided. In one aspect, a method is provided in which enveloped virus present in the product stream is inactivated, while leaving non-enveloped virus intact.

In one aspect, the non-enveloped viral vector is manufactured in a cell culture. In one aspect, the non-enveloped viral vector is harvested from a cell culture. In one aspect, the non-enveloped viral particles accumulate in the host cell cytoplasm and in the cell culture media. In one aspect, the host cells are lysed to increase yield. In one aspect, the host cells are collected, for example, by centrifugation and/or filtration. The collected cells can be frozen and stored or can be further processed for purification of the biotherapeutic. In one aspect, the collected cells are resuspended in buffer to form a cell slurry. In one aspect, the resuspension buffer is selected from Tris-HCl, sodium acetate, potassium citrate, sodium phosphate monobasic or dibasic or mixtures thereof.

In one aspect, the cells in the slurry are contacted with a solution that includes an environmentally compatible detergent. In one aspect, the cells in the cell slurry are contacted with a solution that includes Laureth-9. In one aspect, the cells in the slurry are contacted with a solution that includes Laureth-9 to lyse the cells. In one aspect, the cells in the slurry are contacted with a solution that includes Laureth-9 to inactivate enveloped virus. In one aspect, the solution includes Laureth-9 and one or more additional detergents. In one aspect, the solution includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9. In one aspect, the cells are incubated with the solution containing Laureth-9 for about 1 minute to about 120 minutes. In one aspect, the cells are incubated with the solution containing Laureth-9 for at least about 1, 2, 3, 4, 5, 10, 15, 20, 25 or 30 minutes and up to about 60, 90 or 120 minutes. In one aspect, the mixture is incubated with the solution containing Laureth-9 for up to about 1 hour, 2 hour, 3 hours 4 hours, 5 hours, 24 hours or 48 hours. In one aspect, the cells are incubated with the solution containing Laureth-9 for about 1, 2, 3, 4, 5, 10, 15, 20, 25, 30, 60, 90 or 120 minutes. In one aspect, the mixture is incubated with the solution containing Laureth-9 for about 1 hour, 2 hour, 3 hours 4 hours, 5 hours, 24 hours or 48 hours. In one aspect, the cells are incubated with Laureth-9 for about 1 minute. In one aspect, the cells are incubated with Laureth-9 for at least about 5 minutes. In one aspect, the cells are incubated with Laureth-9 at a temperature from about 4 °C to about 42 °C. In one aspect, the cells are incubated with Laureth-9 at a temperature from about 4 °C to about 25 °C. In one aspect, the cells are incubated with Laureth-9 at room temperature, for example, at a temperature of about 20 °C to about 25 °C. In one aspect, the mixture is contacted with a solution that includes Laureth-9 at a pH from about pH 4.5 to about pH 8.5. In one aspect, the mixture is contacted with a solution that includes Laureth-9 at a pH of about 4.5, 5.0, 5.5, 6.0, 6.5, 7.0, 7.5, 8.0, or 8.5.

In one aspect, titer is determined by the concentration of viral particles containing the viral genome (the physical titer). In one aspect, the physical titer is determined by quantitative PCR (qPCR), digital droplet PCR (ddPCR), or other DNA quantification methods. Physical titer can be expressed in viral genomes per ml (vg/ml). In one aspect, the purified AAV titer is greater than about 10 log₁₀ vg/ml. In one aspect, the physical titer is not reduced compared to a control that is not contacted with Laureth-9. In one aspect, the physical titer is within about 0.05 log₁₀ vg/mL, 0.1 log₁₀ vg/mL, 0.2 log₁₀ vg/mL or 0.3 log₁₀ vg/mL compared to a control that is not contacted with Laureth-9. In one aspect, the control includes cells that were lysed using a freeze-law method. In one aspect, the control includes cells that were lysed using Triton-X.

In one aspect, titer is determined by the concentration of viral particles that can transduce cells (the infectious titer). In one aspect, the infectious titer is quantified by a cell transduction assay, such TCID50 (median tissue culture infectious dose). In one aspect, the purified AAV titer is greater than about 8 log₁₀ TCID50/mL. In one aspect, infectious titer is not reduced compared to a control that is not contacted with Laureth-9. In one aspect, infectious titer is within about 0.1 log₁₀ TCID50/mL 0.2 log₁₀ TCID50/mL 0.3 log₁₀ TCID50/mL, or 0.7 log₁₀ TCID50/mL compared to a control that is not contacted with Laureth-9. In one aspect, the control includes cells that were lysed using a freeze-law method. In one aspect, the control includes cells that were lysed using Triton-X.

In one aspect, the method includes loading a mixture containing the non-enveloped virus and one or more impurities onto a chromatography support; washing the chromatography support with a wash solution that includes Laureth-9; and eluting the non-enveloped virus from the chromatography support to obtain a purified eluate containing the non-enveloped virus. In one aspect, the solution includes Laureth-9 and one or more additional detergents. In one aspect, the solution includes at least about 0.1%, 0.2%, 0.3%, 0.4%, 0.5%, 0.6%, 0.7%, 0.8%, 0.9% or 1% (w/v) and up to 1%, 2%, 3%, 4%, 5% or 10% (w/v) Laureth-9. In one aspect, the solution includes about 0.1%, 0.2%, 0.3%, 0.4%, 0.5%, 0.6%, 0.7%, 0.8%, 0.9%, 1%, 2%, 3%, 4%, 5% or 10% (w/v) Laureth-9.

### INCORPORATION BY REFERENCE

All references cited herein, including patents, patent applications, papers, text books and the like, and the references cited therein, to the extent that they are not already, are hereby incorporated herein by reference in their entirety.

### EXAMPLES

### Example 1. Viral Inactivation Using Non-Ionic Surfactants

The viral inactivation of four detergents was examined using a CHO-derived cell harvest material for a monovalent bispecific human IgG1 monoclonal antibody. Brij L9 (Laureth-9), Myrj S25, and Brij S20 were sourced from Croda International Plc. (East Yorkshire, United Kingdom). Polidocanol 600 (Laureth-9) was sourced from Scharer & Schlapfer AG (Rothrist, Switzerland). Harvest media was spiked with murine xenotropic leukemia virus-related virus (XMuLV) stock prior to the detergent spike.

Conditioned cell harvest media containing a monovalent bispecific human IgG₁ monoclonal antibody (BisAb) was spiked with 8.5 logs of XMuLV. A sample of the virus spiked harvest media was collected and used as a hold control to determine the rate of viral inactivation without detergent.

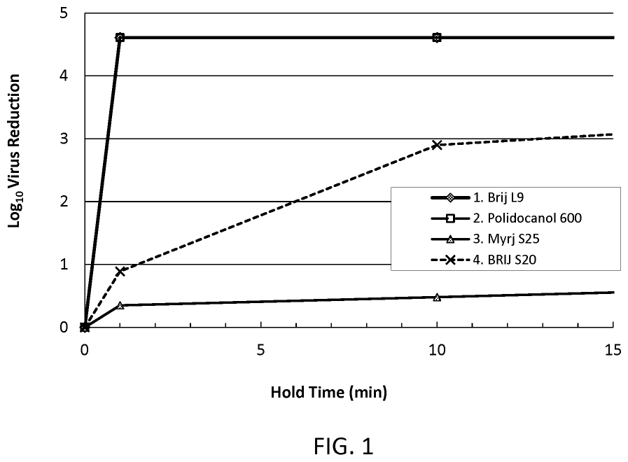

A 10% (w/w) stock solution was made by dissolving detergent into 50 mM Tris pH 7.4. A cell harvest sample of BisAb media spiked with XMuLV was spiked to a 1% detergent concentration using an appropriate amount of the 10% stock solution. The hold control and reaction samples were incubated at room temperature. Samples were collected at 1, 10, 60, and 120 minutes and diluted 1:50 in McCoy medium to stop the viral inactivation reaction. Diluted samples and hold control were assayed for viral concentration using a plaque assay. As shown in Table 1, the virus concentration was reduced from 7.79 to less than 3.18 in less than 1 minute for the Brij L9 and Polidodecanol 600 spiked samples and a greater than 4 Log₁₀ inactivation was observed in 1 minute by detergents Brij L9 and Polidocanol 600 (FIG. 1 and Table 2).

The results shown in Tables 1 and 2 demonstrate that not all structurally similar detergents, or even detergents belonging to the same polyoxyethylene alkyl ether class, will be effective for viral inactivation. Brij S20 (polyoxyethylene (20) stearyl ether), a surfactant that is structurally related to Laureth-9, demonstrated significantly slower and hence significantly less desirable viral inactivation kinetics compared to Laureth-9 as shown in Tables 1 and 2. Only Brij L9 demonstrated improved, desirable viral inactivation kinetics.

TABLE 1
Virus concentration

| Detergent | 0 min | <1 min | 10 min | 60 min | 120 min |
|---|---|---|---|---|---|
| Brij L9 | 7.79 | <3.18 | <3.18 | <3.18 | <3.18 |
| Polidodecanol 600 | 7.79 | <3.18 | <3.18 | <3.18 | <3.18 |
| Myrj S25 | 7.79 | 7.44 | 7.31 | 6.57 | 5.43 |
| Brij S20 | 7.79 | 6.9 | 4.89 | <3.18 | <3.18 |

TABLE 2
Log₁₀ reduction value

| Detergent | 0 min | <1 min | 10 min | 60 min | 120 min |
|---|---|---|---|---|---|
| Brij L9 | 0 | >4.61 | >4.61 | >4.61 | >4.61 |
| Polidodecanol 600 | 0 | >4.61 | >4.61 | >4.61 | >4.61 |
| Myrj S25 | 0 | 0.35 | 0.48 | 1.22 | 2.36 |
| Brij S20 | 0 | 0.89 | 2.90 | >4.61 | >4.61 |

### Example 2. Viral Inactivation Using Laureth-9

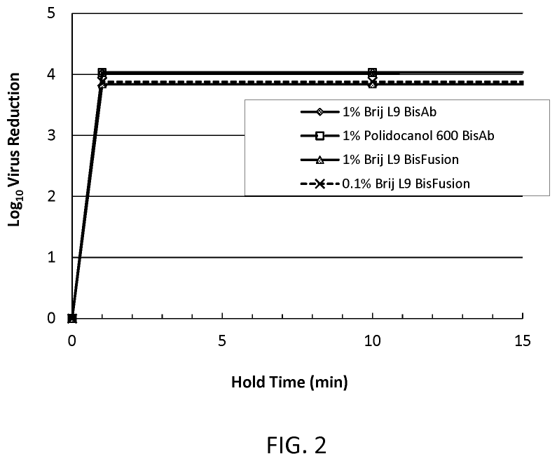

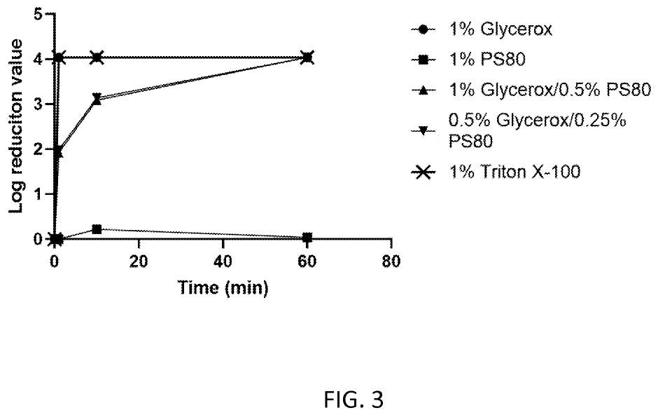

A study was performed essentially as described in Example 1 to examine the viral inactivation using detergent concentrations of 1% Brij L9 (Laureth-9, sourced from Croda International Plc., East Yorkshire, United Kingdom) and 1% Polidodecanol 600 (Laureth-9, sourced from Scharer & Schlapfer AG, Rothrist, Switzerland) for CHO-derived conditioned media for BisAb and 1% or 0.1% Brij L9 for CHO-derived conditioned media for a bispecific fusion protein (BisFusion). As shown in FIG. 2, a greater than 4 Log₁₀ inactivation was observed in all samples in 1 minute. As shown in Table 3, the virus concentration was reduced from 7.00 (BisAb) or 7.21 (BisFusion) to less than 2.88 in less than 1 minute for all four mixtures.

TABLE 3
Virus concentration

| Detergent | Sample | 0 min | <1 min | 10 min | 60 min |
|---|---|---|---|---|---|
| 1% Brij L9 | BisAb | 7.00 | <2.88 | <2.88 | <2.88 |
| 1% Polidodecanol 600 | BisAb | 7.00 | <2.88 | <2.88 | <2.88 |
| 1% Brij L9 | BisFusion | 7.21 | <2.88 | <2.88 | <2.88 |
| 0.1% Brij L9 | BisFusion | 7.21 | <2.88 | <2.88 | <2.88 |

TABLE 4
Log₁₀ reduction value

| Detergent | Sample | 0 min | <1 min | 10 min | 60 min |
|---|---|---|---|---|---|
| 1% Brij L9 | BisAb | 0 | >4.03 | >4.07 | >4.07 |
| 1% Polidodecanol | BisAb | 0 | >4.03 | >4.07 | >4.07 |
| 600 | | | | | |
| 1% Brij L9 | BisFusion | 0 | >3.84 | >3.84 | >3.84 |
| 0.1% Brij L9 | BisFusion | 0 | >3.88 | >3.88 | >3.88 |

### Example 3. Adeno-Associated Virus (AAV) Purification

An AAV6.2 cell paste slurry was generated and stored at −80 °C. Brij L9 (Laureth-9) was sourced from Croda International Plc. (East Yorkshire, United Kingdom). Triton X-100 was sourced from Spectrum Chemical (New Brunswick, NJ). Capto AVB resin was sourced from GE Healthcare (Piscataway, NJ). COSP filters were obtained from EMD Millipore (Burlington, MA). Sartopore 2 XLG, 0.8/0.2 um filters were obtained from Sartorius Stedim Biotech GmbH (Gottingen, Germany). All the buffer salts were sourced from Avantor Performance Chemicals (Center Valley, Pennsylvania).

For detergent lysis, the AAV6.2 cell paste was diluted to a 10% slurry with 20 mM Tris, 200 mM NaCl, 2 mM MgCl2, pH 7.5. 10% (w/w) detergent stock solution was spiked into cell slurry to bring final detergent concentration to 0.5% (v/w). Benzonase was spiked to 10 U/ml into cell slurry mixture and mixed slowly at RT for 1 hour.

For freeze-and-thaw lysis, the cell paste slurry was put through 2 freeze-thaw cycles at −80 °C and RT. AAV lysate was diluted to 10% slurry with 20 mM Tris, 200 mM NaCl, 2 mM MgCl2, pH 7.5. Benzonase was spiked to 10 U/ml into cell slurry mixture and mixed slowly at RT for 1 hour.

AAV Purification was performed using a Capto AVB affinity column. Purification was controlled by Unicorn 7.0 software on an AKTA AVANT purchased from GE Healthcare (Piscataway, NJ). COSP POD filter was flushed with 20 mM Tris, 500 mM NaCl, 2 mM MgCl2, pH 7.5. 10% AAV6.2 cell lysate was clarified with COSP followed by Sartopore 2 XLG, 0.8/0.2 um filter. Capto AVB column was equilibrated with 20 mM Tris, 200 mM NaCl, 2 mM MgCl2, pH 7.5 for 3 column volumes (CV). Clarified cell lysate was loaded with residence time of 6 min. Column was re-equilibrated with 20 mM Tris, 200 mM NaCl, 2 mM MgCl2, pH 7.5 for 4 CV. Product was eluted from column with 50 mM citric acid, 500 mM NaCl, 2 mM MgCl2, pH 2.5. Eluate pH was adjusted to pH 8.0 with 1 M Tris/HCl, pH 8.0

AAV Titer was determined by centrifuging 10% lysate at 13,000 rpm for 10 min and collecting the supernatant. AAV titer was determined with in-house qPCR method.

As shown in Table 5, Brij L9 (Laureth-9) is as effective for cell lysis for AAV purification as Triton X-100

TABLE 5

| | | Lysate AAV6 Titer |
|---|---|---|
| Detergent | AAV6 Cell Paste Lot | (Log₁₀ vg/mL) |
| 0.5% Triton X-100 | A8498 | 11.03 |
| 0.5% Brij-L9 | A8498 | 11.04 |

As shown in Table 6, Brij L9 (Laureth-9) does not show any negative impact on AAV infectivity.

TABLE 6

| | | CaptoAVB | CaptoAVB Elu |
|---|---|---|---|
| | Cell Paste | Elu Titer | Infectivity |
| Cell lysis method | Lot ID | (Log₁₀ vg/mL) | (Log₁₀ TCID50/mL) |
| Detergent Brij-L9 | A8489 | 12.2 | 10.5 |
| Detergent Triton X100 | A8102 | 11.8 | 9.8 |
| Freeze-thaw | A8102 | 12.1 | 10.3 |

### Example 4. Host Cell Protein (HCP) Clearance

A CHO-derived cell harvest material for bispecific antibody was used. Brij L9 (Laureth-9) was sourced from Croda International Plc. (East Yorkshire, United Kingdom). Triton X-100 was sourced from Spectrum Chemicals (New Brunswick, NJ). LambdaFabSelect resin was sourced from GE healthcare (Piscataway, NJ). All the buffer salts were sourced from Avantor Performance Chemicals (Center Valley, Pennsylvania).

A 10% (w/w) stock solution was made dissolving detergent into USP water. Appropriate amount of this stock solution was spiked into the cell harvest material to bring the final detergent concentration to 2% (w/w).

For the chromatography experiment, purification was controlled by Unicorn 7.0 software on an AKTA AVANT purchased from GE Healthcare (Piscataway, NJ). Cell harvest material spiked with detergent was loaded onto LambdaFabSelect column. For wash experiment, cell harvest without spiked detergent was loaded onto the LambdaFabSelect column. Upon protein binding, equilibration was performed using 50 mM Tris, pH 7.4 for 3CV. For wash experiment, a detergent wash containing 1% detergent (w/w) in equilibration buffer was applied on the column for 3CV. This was followed by a 3CV wash of equilibration buffer before eluting the protein from the resin using 25 mM Sodium acetate, pH 3.6 buffer. HCP content in the elution was measured using in-house HCP ELISA assay.

As shown in Table 7, Brij L9 (Laureth-9) performs similar to Triton X-100 and can be used as a wash on affinity chromatography media to remove HCP.

TABLE 7

| | Normalized* | HCP |
|---|---|---|
| Wash Condition | Eluate Yield | (ng/mg) |
| 0.5M NaCl wash | 1.00 | 2853 |
| 1% Brij-L9 | 1.00 | 1924 |
| 2% Brij-L9 | 1.01 | 1646 |
| 1% Triton X-100 | 1.00 | 1856 |

*Eluate yields normalized to 0.5M NaCl wash control condition

As shown in Table 8, Brij L9 (Laureth-9) performs similar to Triton X-100 and can reduce HCP content when spiked into cell harvest material prior to loading on affinity chromatography media.

TABLE 8

| Spike Condition | Normalized Eluate Yield* | HCP (ng/mg) |
|---|---|---|
| No detergent control | 1.0 | 1607 |
| 2% Brij-L9 | 1.0 | 1105 |
| 2% Triton X-100 | 1.0 | 1099 |

*Eluate yields normalized to no detergent spike control condition

### Example 5. Endotoxin Clearance

A CHO-derived monoclonal antibody was used. Lysozyme and Bovine Serum Albumin (BSA) were sourced from Sigma Aldrich (St. Louis, MO). Endotoxin extracted from E. coli serotype O55:B5 was sourced from Sigma Aldrich (St. Louis, MO). Brij L9 (Laureth-9) was sourced from Croda International Plc. (East Yorkshire, United Kingdom). Hi-Trap MabSelectSuRe, Hi-Trap Q HP and Hi-Trap SP sepharose Fast Flow were sourced from GE Healthcare (Piscataway, NJ). Phosphate buffered saline (PBS) was sourced from Sigma Aldrich (St. Louis, MO); all other buffer salts were sourced from Avantor Performance Chemicals (Center Valley, Pennsylvania).

Endotoxin standard was prepared by dissolving 25 mg of 055:B5in 5 mL of USP grade water. This standard was then diluted one hundred times using USP grade water to make the final spiking standard. All protein solutions were spiked to obtain approximately 1000 EU/ml of final endotoxin concentration. All starting endotoxin values were measured and reported.

For the chromatography experiments, purification was controlled by Unicorn 7.0 software on an AKTA AVANT purchased from GE Healthcare (Piscataway, NJ). Protein solutions were spiked with known amount of endotoxin and were bound to various chromatography resins. Upon protein binding, a wash solution of equilibration buffer containing 1% of Laureth-9 was applied to the column over 10 column volumes (CV). This was followed by a 10 CV wash with equilibration buffer before eluting the protein from the resin. Lysozyme-endotoxin material was bound to SP Fast Flow in 1×PBS, pH 7.2 equilibration buffer prior to detergent wash, equilibration, and elution in 1×PBS, 500 mM sodium chloride, pH 7.2. Monoclonal antibodies spiked with endotoxin were bound to MabSelectSuRe in 1×PBS, pH 7.2 prior to detergent wash, equilibration and elution in 50 mM acetate, 30 mM sodium chloride pH 3.5.

Endotoxin concentrations were measured by Endosafe PTS100 with sensitivity to 0.1 EU/mL purchased from Charles River laboratories (Charleston, SC)

Protein concentrations were measured by Nanodrop purchased from Thermo Fisher Scientific (Rockville, MD). The protein concentration of each sample was analyzed by absorbance at 280 nm. After calibrating to zero absorbance with the blank buffer solution, each protein sample was measured for absorbance at A280. The absorbance was divided by the respective extinction coefficient of each protein

As shown in Table 9, Laureth-9 reduces endotoxins to below 1 EU/mg (the typical demand for in-vivo studies) and therefore can be used as a wash on cation exchange chromatography media to remove endotoxins from Lysozyme solution.

TABLE 9

| Wash Condition | Normalized Eluate Yield | Endotoxin (EU/mg) |
|---|---|---|
| Starting endotoxin | | 717 |
| Baseline (no wash) | 1.00 | 21.2 |
| 1% Brij-L9 | 0.8 | 0.39 |

As shown in Table 10, Brij L9 (Laureth-9) reduces endotoxins to below 1 EU/mg which is the typical demand for the in-vivo studies and can therefore be used as a wash on protein-A affinity chromatography media to remove endotoxins from mAb solution.

TABLE 10

| Wash Condition | Normalized Eluate Yield | Endotoxin (EU/mg) |
|---|---|---|
| Starting endotoxin | | 1911 |
| Baseline (no wash) | 1.00 | 179.9 |
| 1% Brij-L9 | 1.02 | 0.88 |

1. A method for purifying a recombinant biotherapeutic of interest produced by a cell culture in a mixture comprising the biotherapeutic of interest, an enveloped virus, and one or more impurities, the method comprising contacting the mixture with a solution comprising a cell disrupting, virus-inactivating and endotoxin removing amount of about 0.1% to about 2% Laureth-9, and incubating the mixture for at least about 1 minute;
wherein at least about 75%, 80%, 85%, 90%, or 95% of the cells in the mixture are lysed after the mixture is incubated with the Laureth-9 solution:
wherein the solution comprising Laureth-9 provides a virus log reduction value (LRV) greater than about 1 log₁₀, 2 log₁₀, 3 log₁₀ or 4 log₁₀ or more as compared to a control that does not include a surfactant; and
wherein the endotoxin concentration is reduced to less than about 1 EU/mg.

2-7. (canceled)

8. The method according to claim 1, wherein the enveloped virus comprises a DNA or RNA virus which comprises a single stranded or double stranded virus.

9. (canceled)

10. The method according to claim 1, wherein the enveloped virus includes Herpesviridae, Hepadnaviridae, Togaviridae, Arenaviridae, Flaviviridae, Orthomyxoviridae, Paramyxoviridae, Bunyaviridae, Filoviridae, Coronaviridae, Bornaviridae, Arteriviridae, or a combination thereof.

11-14. (canceled)

15. The method of claim 1, wherein the mixture is incubated with Laureth-9 for about 1 minute to about 120 minutes.

16. The method of claim 1, wherein the mixture is incubated with Laureth-9 at room temperature.

17-18. (canceled)

19. The method of claim 1, wherein the solution comprises from about 1% to about 2% (w/v) Laureth-9.

20. The method of any of claim 1, further comprising a step of filtering the mixture comprising the recombinant biotherapeutic of interest.

21. The method of claim 20, wherein filtering is performed before incubating the mixture with the solution comprising Laureth-9 or after incubating the mixture with the solution comprising Laureth-9.

22. (canceled)

23. The method of claim 20, wherein filtering comprises ultrafiltration or depth filtration.

24. The method of claim 1, further comprising one or more chromatography steps comprising loading a mixture comprising the biotherapeutic of interest onto a chromatography support and washing the chromatography support with a wash solution comprising Laureth-9.

25. (canceled)

26. A method for purifying a biotherapeutic of interest, the method comprising:
(a) loading a mixture comprising the biotherapeutic of interest and one or more impurities onto a chromatography support;
(b) washing the chromatography support with a wash solution comprising a host cell protein-clearing, virus-inactivating and endotoxin removing amount of Laureth-9 of about 0.1% to about 2% (w/v) Laureth-9; and
(c) eluting the biotherapeutic of interest from the chromatography support to obtain a purified eluate comprising the biotherapeutic of interest,
wherein the purified eluate comprises a host cell protein content that is reduced relative to a host cell protein content in an eluate from a chromatography support that was not washed with a wash solution comprising Laureth-9;
wherein the host cell protein content in the purified eluate from the chromatography support was reduced by at least about 50%, 45%, 40%, 35%, 30% or 25% as compared to the host cell content of the mixture applied to the column;
wherein the host cell protein content in the purified eluate from the chromatography support is less than about 2000 ng/mg, 1700 ng/mg, 1500 ng/mg, 1200 ng/mg, 1000 ng/mg, 500 ng/mg, or 100 ng/mg; and
wherein the purified eluate comprises an endotoxin concentration below about 1 EU/mg.

27-34. (canceled)

35. The method of claim 1, wherein the recombinant biotherapeutic of interest is a therapeutic polypeptide, a therapeutic polynucleotide, an enzyme, a soluble receptor, a growth factor, a hormone, a cytokine, an antibody, an antigen-binding antibody fragment, an antibody-drug conjugate, a fusion polypeptide.

36. (canceled)

37. The method of claim 35, wherein the antibody is a monoclonal antibody, polyclonal antibody, multivalent antibody, multispecific antibody, chimeric antibody, humanized antibody or human antibody.

38-41. (canceled)

42. The method of claim 1, wherein the recombinant biotherapeutic of interest is produced by a mammalian cell.

43. The method of claim 42, wherein the mammalian cell comprises Chinese Hamster Ovary (CHO), baby hamster kidney (BHK), murine hybridoma, or murine myeloma cells.

44. The method of claim 26, wherein chromatography comprises one or more of affinity chromatography, ion exchange chromatography hydrophobic interaction chromatography, hydroxyapatite chromatography, and mixed mode chromatography.

45. The method of claim 44, wherein chromatography comprises affinity chromatography selected from protein A chromatography, protein G chromatography or protein L chromatography.

46. (canceled)

47. The method of claim 44, wherein ion exchange chromatography is selected from anion exchange chromatography and cation exchange chromatography.

48. The method of claim 26, further comprising a step of filtering a product stream.

49. The method of claim 48, wherein filtering comprises ultrafiltration or depth filtration.

50-67. (canceled)
